(19) United States
(12) Patent Application Publication
(10) Pub. No.: US 2009/0238853 A1
(43) Pub. Date: Sep. 24, 2009

(54) Hybrid Biomedical Device Fabricated From Biomaterials and Coated With a Natural Extra Cellular Matrix (ECM) Coating

(75) Inventors: Qing Liu, Hillsborough, NJ (US); Wing K. Lau, Basking Ridge, NJ (US); Marika Katherine Bergenstock, North Plainfield, NJ (US)

Correspondence Address: Qing Liu, 55 Nostrand Road, Hillsborough, NJ, 08844, US

(73) Assignee: 3D BIOTEK, LLC, North Brunswick, NJ (US)

(21) Appl. No.: 12/408,454

(22) Filed: Mar. 20, 2009

Related U.S. Application Data
(60) Provisional application No. 61/038,538, filed on Mar. 21, 2008.

(51) Int. Cl. C12N 5/08; A61F 2/02; C12N 5/00; C12N 5/06

(52) U.S. Cl. 424/423; 435/402; 435/325; 435/366; 435/404; 435/408

(57) ABSTRACT

The present invention relates to a hybrid biomedical device formed from a prefabricated biomedical device and coated with an extracellular matrix coating which is produced by directly culturing mammalian cells on the prefabricated biomedical device. The purposes of applying such an ECM coating is to regulate the biological and cellular responses of a living tissue or a cellular environment that the hybrid biomedical device is intended to interact with. The ECM coating will provide necessary biological cues, biomechanical cues, structural cues, etc to regulate the biological and cellular responses of a living system. The invention further provides methods of making the hybrid biomedical device.

### CROSS-REFERENCE TO RELATED APPLICATION: PROVISIONAL APPLICATIONS 61/038,538

The present application claims benefit of provisional applications: 61/038,538; the disclosure of which is hereby incorporated by reference.

### SEQUENCE LISTING

None

### 1. FIELD OF THE INVENTION

The present invention relates to a biomedical device with an extracellular matrix (ECM) coating for use as a surgical implant, tissue engineering scaffold and tissue/cell culture substrate. The primary biomedical device which will bear the ECM coating can be fabricated from metals, ceramics, polymers, naturally derived biomaterials or composites of these materials. The biological ECM coatings are produced by culturing living mammalian cells on the surfaces of said devices. During the culturing process, the living cells secrete and lay down ECM on the surfaces of the devices, implants and scaffolds. As a result, this ECM coating is composed of a range of major ECM proteins and other biological components, such as growth factors and cytokines. These ECM molecules and factors not only have their natural structure, but also have the natural conformation for them to perform their biological function. When the cells are deemed finished with the ECM coating process, the living cells are removed by a de-cellularization process or are destroyed by physical-chemical means, such as lyophilization. The final hybrid medical device with ECM coating is stored under dehydrated or frozen conditions in order to preserve the native biological structure and composition of the ECM coating. The biomedical device could be used with/without adding heterologous/autologous living cells when used in repairing human tissue defect. This device could also be used as an in vitro cell culture device for living cells to attach, proliferate and differentiate.

### 2. BACKGROUND OF THE INVENTION

Utilizing medical devices, surgical implants and tissue engineering scaffolds fabricated from biomaterials to repair various human tissue defects and restore normal tissue/organ functions is a common practice nowadays. Although the surfaces of these implants/scaffolds are “biocompatible” and “non-cytotoxic”, these implant/scaffold surfaces are not physiologically relevant to cells or living tissue. Therefore, artificial surgical implants that are currently in use today are still not ideal because they frequently elicit a foreign body reaction and do not integrate well with the surrounding tissue. An ideal implant or tissue engineering scaffold should promote cell adhesion, proliferation, and tissue regeneration, or wound healing when the host's tissue, or defect, needs to be repaired. Most biomaterials used nowadays are not ideal in terms of promoting wound healing and tissue regeneration. Therefore, the tissue repairing or wound healing process is slow. Patients will likely have a greater chance of infection, a longer recovery process, and an increased chance for a recurring injury.

On the other hand, naturally derived de-cellularized ECM from tissue/organs performs better in terms of promoting wound healing and integration with surrounding tissue. Examples of these tissues are, human amniotic membrane¹, porcine small intestine submucosa (SIS)², decellularized human dermis Alloderm®³, and heart valve⁴. These naturally derived ECMs, after being processed and decellularized, contain many extracellular matrix components such as collagen, fibronectin, laminin, glycosaminoglycans, and numerous other biological molecules including growth factors such as transforming growth factor (TGF). Clinical and experimental studies have shown that these naturally derived ECM are superior implants/scaffolds in terms of in vivo biological performance.

The superior biological performance of this naturally derived ECM can be attributed to its composition and origin. ECM is the product of the resident cells of each tissue and organ and has both structural and functional roles. The ECM is in a state of dynamic equilibrium with its adjacent cell population and is also highly responsive to ever changing physiological environment as well as to the functional demands of the neighboring cells and their parent tissue or organ⁵. ECM is a dynamic, multifunctional structure that is composed of a complex mixture of proteins, proteoglycans, and glycoproteins, with diverse, but tissue-specific composition and organization. Some of the important in vivo functions of the ECM include maintenance of cell structure and function, tissue and organ morphogenesis, and wound healing⁶. Among major ECM proteins, collagen is the most abundant protein. More than 20 distinct types of collagen have been identified and the most prevalent form found in mammalian ECM is type I collagen. Fibronectin represents an important non-collagenous component of the ECM. Fibronectin exists both in soluble and tissue isoforms and possesses many desirable properties of a tissue repair scaffold, including ligands for adhesion of many cell types^{7,8}. Laminin is another complex adhesion protein found within the ECM, especially within the basement membrane form of ECM⁹

Besides the major ECM proteins mentioned above, ECM also includes a variety of bioactive molecules admixed with the binding molecules such as decorin and biglycan. Growth factors and cytokines are also found in ECM. Although cytokines and growth factors are present within ECM in extremely small quantities, these molecules act as potent modulators of cell behavior. There is a long list of growth factors that can be found within the ECM, that includes VEGF, bFGF, EGF, transforming growth factor-β (TGF-β), keratinocyte growth factor (KGF), hepatocyte growth factor (HGF), and platelet-derived growth factor (PDGF). These growth factors tend to exist in multiple isoforms, each with a specific biologic activity¹⁰.

The presence of the multiple isoforms of cytokines and growth factors suggests that the tissue growth needs multiple, and very specific growth factors. Researchers have used purified forms of cytokines, growth factors, and active biological peptides as therapeutic means of encouraging blood vessel formation (vascular endothelial cell growth factor, VEGF), inhibiting blood vessel formation (Angiostatin), inducing bone formation (bone morphogenetic protein, BMP), stimulating deposition of granulation tissue (platelet-derived growth factor, PDGF), and encouraging epithelialization of wounds (keratinocyte growth factor, KGF). However, obstacles have been encountered that include determining optimal dose, achieving a sustained and localized release of the growth factor at the desired site, and turning the factor on and off as needed during the course of tissue repair. Therefore, some researches have suggested that it may be an advantage to use naturally derived ECM as a scaffold for tissue repair because of the presence of all of attendant growth factors (and their inhibitors) in the relative amounts that exist physiologically and, perhaps most importantly, in their native three-dimensional ultra-structure¹⁰.

In addition to the biochemical cues or signals that ECM provides, the ECM also provides the principal means by which mechanical information is communicated between tissue and cellular levels of function. These mechanical signals play a central role in controlling cell fate and establishing tissue structure and function.

Mechanical loads have been identified as critical determinants of cell behavior not only in vivo, during physiological and pathological processes, but also in vitro for engineering functional tissue constructs¹¹. These mechanical signals may be evoked via the contractile machinery of the resident cells or by a variety of environmental factors¹². The process by which these physical forces are converted into biochemical signals and integrated into cellular responses is referred to as mechanotransduction¹³. A critical component of the mechanotransduction process is the extracellular matrix (ECM) and its interface with resident cells. The ECM interacts with cells to provide relevant micro-environmental information; biochemically, through soluble and insoluble mediators and physically, by imposing structural and mechanical constraints. Mechanical cues present in the ECM have been hypothesized to provide instructive signals that dictate cell behavior. It has been shown that culturing osteoblastic MC3T3-E1 cells on the surface of type I collagen modified hydrogels with tunable mechanical properties stimulates different cell behavior. On gels functionalized with a low type I collagen density, MC3T3-E1 cells cultured on polystyrene proliferated twice as fast as those cultured on the softest collagen substrate¹⁴.

The 3-dimensional (3D) molecular composition and complex microstructure organization of ECM also plays an important role in cellular interaction. However, the physiological relevance of such cell-ECM signaling as it occurs within a context of 3D structural-mechanical complexity is just being realized. This critical role played by ECM microstructure is largely owing to the fact that it controls how mechanical loads are transferred between a cell and its microenvironment as part of the mechanotransduction process. It is well documented that, compared with fibroblasts grown in a 2D format, those grown within 3D collagen matrices develop a morphology that is more reminiscent of that observed in vivo^{15,16}.

In summary, ECM is an ideal 3D matrix that provides both biological and mechanical cues through its complex 3D microstructure to regulate local cell-ECM biomechanics and fundamental cell behavior.

ECM is now well known to have the ability to regulate virtually all cell functions, including adhesion, spreading, migration, proliferation, survival, and differentiation. Thus far no man-made biomaterials can achieve such a complicated level of control or regulation on cellular behaviors. Therefore, ECM is an ideal substrate to support and promote key cellular functions. Indeed, naturally derived decellularized tissue/organ materials perform much better in terms of promoting wound healing, tissue remodeling and integration with surrounding tissue.

However, decellularized ECM derived from soft tissue often have poor mechanical properties compared with man-made biomaterials, such as metals and polymers, especially if the implant needs to be used in load-bearing applications. Also, decellularized ECM from soft tissue does not have the same capability to be processed into desired sizes and shapes as synthetic polymers and metals, which are known to be easily processed into various sizes and shapes via modern processing techniques such as casting, molding, injection molding, and extrusion, etc. Furthermore, ECM derived from human or animal cadaver also carries the risk for disease transmission and lacks of consistent quality due to inherent donor variation. These constraints greatly limit the use of ECM in many applications.

To take advantage of the both superior biological performance of ECM and the processing ability of synthetic biomaterials, we have developed a hybrid biological ECM coated biomedical device. The biomedical device will be first fabricated using an appropriate processing method and subsequently used as a substrate for culturing mammalian cells on its surface to obtain an ECM coating layer that was synthesized and secreted by the cultured cells in culture conditions. Upon completion of the cell culture process, the living cells are removed through de-cellularization processes, or destroyed by a physical-chemical process such as lyophilization. The prefabricated device provides the bulk properties such as size, shape, and porosity, while the naturally derived ECM coating on the surface provides the biological performance to promote the cell attachment, proliferation and differentiation to facilitate the neo-tissue formation or tissue remodeling around the biomedical device. These naturally derived ECM coatings on surgical implants greatly improve wound healing and the implant-tissue integration process. If the ECM coating is used on tissue engineering scaffolds, these ECM coated scaffolds will have improved cell loading efficiency and a means to modulate the cellular growth and differentiation.

Therefore, it is the purpose of this invention to develop a hybrid implant/scaffold, which is composed of an implant/scaffold made from biomaterials and a biologically derived ECM coating. The biomaterials can be metals, ceramics, polymers, and naturally derived biomaterials or a composite out of these biomaterials. The ECM coating can be produced by in vitro culture method. For example, the chosen cell lines can be seeded onto the surface of, or onto the pores of the porous scaffolds, to produce an ECM coated biomedical device for surgical implantation, in vitro/in vivo tissue regeneration, and tissue/cell culture use.

One of the advantages of creating such an ECM coating by such cell culture methods is that it offers a very versatile method to control the biological composition of the coating in order to achieve the best performance for its intended use. It is known that different cell lines under different cell culture conditions will have different preferences to secret certain types of ECM components and biological factors. Therefore, by choosing the appropriate cell line for culture, the ECM composition and the factors secreted by the cells could be tailored for different applications. For example, fibroblast culture will produce a ECM layer containing structural extracellular matrix proteins (collagen, fibronectin, etc.) and angiogenic growth factors (including VEGF, bFGF, and HGF) which have been shown to stimulate angiogenic activity¹⁷. Therefore, this type of ECM may be very useful for use in areas where rapid vascularization or angiogenesis is needed.

Another example is that osteoblast culture could produce mineralized bone-like ECM which is always accompanied by an increased level of alkaline phosphatase (ALP). Several studies have shown that the presence of ALP had greatly enhanced the mineralization process of collagen ECM matrix both in vitro and in vivo¹⁸. It is well known that mineralization is an important process in forming new bone. Therefore, the mineralized ECM coating produced by osteoblast culture is very useful in promoting new bone formation and implant/bone integration.

ECM coating with certain selected growth factors can be produced via gene delivery/engineering methods. Cells used in producing ECM coating can be genetically modified to introduce certain DNA fragments so the cells will specifically produce the interested growth factors. For example, human periosteum-derived cells can be transfected with BMP-2 and VEGF genes. These transfected cells have shown to produce increased levels of BMP-2 and VEGF secretion in cell culture¹⁹. It is known that physiologically, BMP-2 is present in bone tissue as matrix-bound form, interacting with extracellular matrix proteoglycans. Therefore, the BMP-2 produced by this cell culture method will be matrix bound in a natural and active form. This matrix bound BMP-2 will retain its bioactivity in a way similar to that of BMP-2 in demineralized bone matrix. Therefore, compared to the delivery of purified BMP-2, which is needed in much higher doses to be effective, to a bone defect to accelerate bone growth rate, this matrix bound BMP-2 will not have the side effect that was caused by the higher dose of purified BMP delivery. It will be a much safer and effective way for bone repair.

Similarly, native VEGF is also matrix bound. A study showed that although free form VEGF is a potent angiogenic stimulus, the blood vessels formed by exposure to free form VEGF tend to be malformed and leaky. Where the matrix bound VEGF induces blood vessel formation more potently than free form VEGF and that those vessels possess more normal morphologies and the vessels induced by bound VEGF do not leak²⁰.

Co-culture technique is also useful in producing the ECM coating. It has been demonstrated that co-culture can produce more sophisticated tissue like structure. By co-culture of human skin fibroblasts and endothelial cells (ECs) from the human umbilical vein (HUVECs), a complex 3D network with EC tubular structures, lumen formation, pinocytotic activity, and tight junction complexes have been identified with the tubular networks extended up to 400 μm²¹. Tissue like structure created using such a co-culture technique provides additional benefit in promoting neo-vascularization within the ECM coating or tissue engineering scaffolds, as vascularization of scaffolds for tissue repair is one of the rate-limiting steps in the field of regenerative medicine.

Decellularization of the cultured cells from cell culture derived ECM coating is achieved using commonly available methods, such as trypsin-EDTA treatment (a common way to remove attached cells from cell culture plates and vessel walls), detergent washing²², and EDTA solution treatment, etc.

Because the cells used in the culture to produce the ECM will be decellularized or destroyed, there will be no risk of living cell provoked immune reaction or concerns of tumorgenesis in the case that live stem cells are involved.

Animal derived cells can also be used to produce the ECM coating. It is known that the basic components of the ECM show a large degree of conservation among species with regard to molecular composition. In other words, the major structural proteins such as collagen, and the adhesive molecules including the glycosaminoglycans, proteoglycans, and glycoproteins, are remarkably similar in their basic amino acid structure and molecular structure between species¹⁰. Because of composition and structure similarity of major ECM proteins, animal derived ECM, such as bovine pericardium, ECM biologic scaffold materials derived from the porcine small intestinal submucosa (SIS) and porcine urinary bladder submucosa (UBS) have been successfully used as surgical implants and tissue engineering scaffolds. The benefits of using such a biomedical device with a cell culture produced ECM coating are:
1. This cell culture derived ECM has more complete natural ECM components. Not only does it contain ECM proteins, but it also contains other components such as growth factors and cytokines. These proteins and growth factors will retain their original structure and conformation when properly processed and preserved. Therefore, this ECM coating is a better coating than single ECM component coatings, such as collagen, fibronectin, or laminin coating.
2. The control of ECM coating composition and biological performance can be achieved by using different cell lines.
3. The ECM coating has no living cells. This will make the storage, transportation and regulatory approval much easier.
4. The coating can be terminally sterilized using e-beam, γ-radiation, or ethylene oxide, or it can be produced in a sterile environment.
5. Unlike ECM derived from cadaver, the cell culture derived ECM is much safer in terms of disease transmission, because the ECM coating comes from cultured cell lines, which have been and can be strictly tested before use.
6. Unlike cadaver derived ECM, in which the quality of the ECM will vary from donor to donor, cell culture produced ECM coating can have consistent quality because the manufacturing conditions can be tightly controlled in culture conditions.
7. The coating can be applied to wide range of materials with various mechanical properties to meet the application requirements.
8. The bioactivity, the composition, and microstructure can be controlled by choosing different cell culture conditions (culture time, culture medium composition, etc), cell lines (cell type, single cell line or co-culture of two or more cell lines) for different applications.
9. This ECM coating is biodegradable, therefore a totally biodegradable implant or scaffold can be created by applying such a coating to a biodegradable implant/scaffold.
10. Since the components of ECM structure among different species is conserved, animal cells can be used in culture to create such an ECM coating. This means that cells available for producing the ECM coating are virtually unlimited, even if animal embryonic stem cells are needed.

### 3. SUMMARY OF THE INVENTION

### 3.1 Hybrid Biomedical Device

It is therefore an objective of the present invention to provide a hybrid biomedical device which consists of a prefabricated device coated with an ECM coating produced by direct culturing of mammalian cell on the surface of the device. The prefabricated device can be made from any type of materials, such as ceramics, metals, synthetic polymers, naturally derived biomaterials or a composite form of these materials. The prefabricated device can have a porous or none porous structure.

The hybrid biomedical device is either dehydrated after being decellularized or stored in a frozen condition. The dehydration process can be a lyophilization process or an organic solvent dehydration process using water miscible organic solvent gradient, such as alcohol solvent gradient.

In one embodiment, the ECM coating can have a porous structure which is produced by freeze-drying or a lyophilization process.

In one embodiment, the biomedical device has a porous structure which will allow cells to get within the pores as well as grow within these pores during the cell culture process.

In one embodiment, the biomedical device has a rough surface to allow for better adhesion between the device and the cells and the ECM coating produced by the cells.

In one embodiment, the biomedical device has a pre-existing coating which facilitates the attachment of the cells that will be used to produce the ECM coating. Said pre-existing coating is a naturally derived proteins, such as collagen, fibronectin. Said pre-existing coating is either chemically bound or physically absorbed onto the biomedical device.

The prefabricated biomedical device can also be surface treated by physical-chemical means, such as plasma surface treatment, to facilitate cellular adhesion.

In one embodiment, the biomedical device is made from metal, such as titanium, and its alloys.

In one embodiment, the biomedical device is made from biodegradable polymers such as polycaprolactone, polylactide, etc.

In one embodiment, said biomedical device is made from ceramics, such as tricalcium phosphate, hydroxyapatite, Bioglass, and Al₂O₃.

In one embodiment, said biomedical device is a composite made from two or more different materials, such as hydroxyapatite/polycaprolactone composite.

In one embodiment, the biomedical device is a porous tissue engineering scaffold made from biodegradable polymers, such as polycaprolactone and polylactic acid.

In one embodiment, therefore, the invention provides a hybrid 3D porous scaffold where the ECM coating is created within the porous structure of the biomedical device.

In a specific embodiment, the prefabricated biomedical device is impregnated with one or more bio-molecules. A bio-molecule can be a protein, peptide, glycosaminoglycan, a naturally occurring compound or polymer, a therapeutic agent, or a combination thereof.

In one embodiment, the cells cultured on the biomedical device are from a human cell line, such as human dermal fibroblast, human umbilical vein endothelia cell (HUVEC), or human bone marrow mesenchymal stem cells

In one embodiment, the cells cultured on the biomedical device are from two or more human cell lines, such as the co-culture of human dermal fibroblast and human umbilical vein endothelia cell (HUVEC).

In one embodiment, the cells cultured on the surface of this biomedical device are of a mixed cell populations, such as bone marrow.

One method of growing cells on three dimensional biomedical devices is to immerse the device in a cell suspension within a spinner flask, and then placing the flask in an incubator appropriate for cellular maintenance. The cells in suspension are then allowed a sufficient period of time to attach to the biomedical device before submerging the biomedical device in a growth medium inside a cell culture apparatus such as a cell culture plate, dish or bioreactor.

### 4. BRIEF DESCRIPTION OF THE FIGURES

FIG. 1. An example of porous biomedical device constructed from polycaprolactone.

FIG. 2. Extracellular matrix secreted by human mesenchymal stem cell culture during osteogenic differentiation within porous polycaprolactone (PCL) scaffold at day 7.

FIG. 3. Von Kossa staining of mouse osteoblasts 7F2 in cultured polycaprolactone (PCL) scaffold on day 7 of culture. No mineralization of the ECM coating was observed on day 7.

FIG. 4. Von Kossa staining of cultured mouse osteoblasts 7F2 in polycaprolactone (PCL) scaffold on day 28 of culture. The dark brown-black staining shows positive result of osteoblast mineralization (of the ECM coating) on day 28.

FIG. 5. An example of a porous biomedical device constructed from polystyrene.

FIG. 6. NIH-3T3 fibroblasts were cultured on porous polystyrene scaffold at day 7. Cell nuclei were stained with DAPI (blue).

FIG. 7. NIH-3T3 fibroblasts were cultured on porous polystyrene scaffold at day 7. F-actin microfilaments were stained with AlexaFluor 488 phalloidin (green).

### 5. DETAILED DESCRIPTION OF THE INVENTION

The present invention provides a hybrid biomedical device which consists of a prefabricated device and an extracellular coating produced by an in vitro cell culture process. The biomedical device provides an internal and external space for cellular adhesion and growth. ECM coating is then secreted by the cells during their growth processes. This cell culture produced ECM has more complete natural ECM components. Native ECM not only contains ECM proteins, but also contains other important matrix components such as growth factors and cytokines. These proteins and growth factors will retain their original structure and conformation when properly processed and preserved. Therefore, this ECM coating is a better coating than a single ECM component coating for biomedical use in promoting cell migration, attachment, proliferation and differentiation. This feature will render the hybrid biomedical device as very useful in vitro cell culture devices, implants and tissue engineering scaffolds.

### 5.1 Configurations

The configuration of the hybrid biomedical device is largely determined by the configuration of the prefabricated biomedical device used in the ECM coating process. The hybrid biomedical device described in the present invention may be configured to any size, and shape to accomplish the particular purpose at hand, e.g., size and shape, which suits its particular application.

The hybrid biomedical device described in the present invention may be porous with any pore size and porosity to accomplish the particular purpose at hand.

In one embodiment, therefore, the invention provides a hybrid 3D porous scaffold where the ECM coating is created within the porous structure of the biomedical device

In one embodiment, therefore, the invention provides a 3D hybrid structure which is composed of a 3D porous disc shape polymer scaffold and an ECM coating.

In one embodiment, therefore, the invention provides a 3D hybrid structure which is composed of a metallic implant, such as a titanium hip implant, a dental implant, and an ECM coating.

In one embodiment, therefore, the invention provides a 3D hybrid device which is composed of a porous 3D cylindrical scaffolds and an ECM coating.

In one embodiment, therefore, the invention provides a 3D hybrid device which is composed of porous 3D tubular scaffolds and an ECM coating.

In one embodiment, therefore, the invention provides a 3D hybrid device which is composed of a tube and an ECM coating.

In one embodiment, therefore, the invention provides a hybrid expandable stent which is composed of an expandable metallic stent and an ECM coating.

In one embodiment, therefore, the invention provides a hybrid expandable stent witch is composed of an expandable polymer stent and an ECM coating.

In one embodiment, therefore, the invention provides a hybrid implant which is composed of an irregularly shaped, prefabricated device with an ECM coating.

In one embodiment, therefore, the invention provides hybrid micro beads which are composed of various sizes of micro beads with ECM coating.

In one embodiment, therefore, the invention provides hybrid micro particles which are composed of various sizes of micro particles with ECM coating.

In a specific embodiment, said hybrid biomedical device is a 3 dimensional disc-shaped porous structure. In another specific embodiment, said cell culture construct is a cubic, 3 dimensionally shaped porous structure.

In one embodiment, therefore, the invention provides a hybrid implant which is composed of a prefabricated device with an ECM coating. In addition, the prefabricated biomedical device has a rough surface to improve the attachment of ECM coating.

In a specific embodiment, said prefabricated biomedical device has pores of constant size and/or dimension, or pores of variable size and/or dimension. In addition, the biomedical device can have pores of constant size and/or dimension for each plane, but the pores on each plane are different from plane to plane in terms of size and/or dimension. Alternatively, the change in pore size and/or dimension can just be one or a few pores on a plane relative to pores on other planes. Further, the size and/or dimension for the pores on each plane could decrease or increase in size.

### 5.1.1 Dimensions

The dimensions of said hybrid biomedical device are primarily determined by the dimensions of the prefabricated biomeidical device. The prefabricated biomedical device in this invention may be pre-fabricated to standard sizes, or may be custom-made to fit into a particular cell culture well, plate, chamber, flask, or bioreactor. In one embodiment, therefore, the invention provides a hybrid biomedical device with a size (both diameter and height) that fits into a round well of a tissue culture plate that is commercially available. In another embodiment, the invention provides a hybrid biomedical device with a cubic shape and size (length×width×height) that fits into a rectangular well of a tissue culture plate. In another embodiment, the hybrid biomedical device has a size and shape that fits into a chamber of a bioreactor. In a specific embodiments, the size of the hybrid biomedical device fits into a tissue culture flask.

### 5.2 Cells for Producing ECM Coating

In one embodiment, the cells cultured on the biomedical device are from a human cell line, such as, but not limited to, human dermal fibroblast, human umbilical vein endothelia cell (HUVEC), human bone marrow mesenchymal stem cell.

In one embodiment, the cells cultured on the biomedical device are from two or more human cell lines, such as co-culture of human dermal fibroblast and human umbilical vein endothelia cell (HUVEC).

In one embodiment, the cells cultured on the surface of biomedical device are a mixed cell population, such as these found in bone marrow.

In one embodiment, the cells cultured on the biomedical device are from a non-human source, such as porcine, bovine, etc.

### 5.3 Materials

In one embodiment, said hybrid biomedical device is composed of a prefabricated biomedical device, and an ECM coating. The said prefabricated biomedical device is made from metal, such as titanium and its alloy, etc.

In one embodiment, said prefabricated biomedical device is made from non-degradable polymer such as polyethylene, polyether ether ketone (PEEK), etc.

In one embodiment, said prefabricated biomedical device is made from biodegradable polymer such as polycaprolactone, polylactic acid, etc.

In one embodiment, said prefabricated biomedical device is made from ceramics, such as tricalcium phosphate, hydroxyapatite, Bioglass, and Al₂O₃, etc.

In one embodiment, said prefabricated biomedical device is made from naturally derived biomaterials, such as chitosan, collagen, alginate, gelatin, cellulose, etc.

In one embodiment, said prefabricated biomedical device is a composite made from two or more different materials, such as hydroxyapatite/polycaprolactone composites, tricalcium phosphate/gelatin composites, hydroxyapatite coated titanium hip implant, etc.

In one embodiment, the biomedical device has a porous structure which will allow cells to get into the pores and grow within the pores during the cell culture process.

In one embodiment, the biomedical device has a rough surface to allow a better adhesion between the device and the cells, and the ECM coating produced by the cells.

In one embodiment, the biomedical device has a pre-existing coating which will facilitate the attachment of the cells that will be used to produce the ECM coating. Said pre-existing coating is naturally derived and is composed of proteins, such as collagen and fibronectin. Said pre-existing coating is either chemically bound or physically absorbed onto the biomedical device.

The prefabricated biomedical device can also be surface treated by physical-chemical means, such as plasma surface treatment, to facilitate cellular adhesion.

The prefabricated biomedical device in the present invention is made of a composite, such as a polymer/polymer composite, polymer/ceramic composite, metal/ceramic composite, and polymer/metal composite.

In a specific embodiment, the prefabricated biomedical device is impregnated or coated with one or more bio-molecules. A bio-molecule can be a protein, peptide, glycosaminoglycan, a naturally occurring compound or polymer, a therapeutic agent or a combination thereof.

The hybrid biomedical device can be further impregnated or coated with one or more biomolecules. A biomolecule can be a protein, peptide, glycoaminoglycan, a naturally occurring compound or polymer, a therapeutic agent or a combination thereof. Examples of naturally occurring compounds or polymers are collagen, laminin, or fibronectin. Therapeutic agents include, but are not limited to, antibiotics, hormones, growth factors, anti-tumor agents, anti-fungal agents, anti-viral agents, pain medications, anti-histamines, anti-inflammatory agents, anti-infective, wound healing agents, wound sealants, cellular attractants, cytokines and the like. A therapeutic agent is anything that when applied would benefit human health. Antibiotics are chemotherapeutic agents that inhibit or abolish the growth of micro-organisms, such as bacteria, fungi, or protozoans. Examples of common antibiotics are penicillin and streptomycin. Other known antibiotics are amikacin, gentamicin, kanamycin, neomycin, netilmicin, tobramycin, paromomycin, geldanamycin, herbimycin, loracarbef, ertapenem, doripenem, imipenem/cilastatin, meropenem, cefadroxil, cefazolin, cefalotin or cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefdinir, cefepime, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, nafcillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, mafenide, prontosil, sulfacetamide, slfamethizole, slfanilimide, sulfasalazine, sulfisoxazole, trimethoprim, trimethoprim-sulfamethoxazole, demeclocycline, doxycycline, minocycline, oxytetracycline, tetracycline, arsphenamine, chloramphenicol, clindamycin, lincoamycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin or rifampicin and tinidazole.

A hormone is a chemical messenger that carries a signal from one cell (or group of cells) to another via the blood. Examples of hormones are melatonin, serotonin, thyroxine, triiodothyronine, epinephrine, norepinephrine, dopamine, antimullerian hormone, adiponectin, adrenocorticotropic hormone, angiotensinogen and angiotensin, antidiuretic hormone, atrial-natriuretic peptide, calcitonin, cholecystokinin, corticotropin-releasing hormone, erythropoietin, follicle-stimulating hormone, gastrin, ghrelin, glucagon, gonadotropin-releasing hormone, growth hormone-releasing hormone, human chorionic gonadotropin, human placental lactogen, growth hormone, inhibin, insulin, insulin-like growth factor, leptin, luteinizing hormone, melanocyte stimulating hormone, oxytocin, parathyroid hormone, prolactin, secretin, somatostatin, thrombopoietin, thyroid-stimulating hormone, thyrotropin-releasing hormone, cortisol, aldosterone, testosterone, dehydroepiandrosterone, androstenedione, dihydrotestosterone, estradiol, estrone, estriol, progesterone, calcitriol, calcidiol, prostaglandins, leukotrienes, prostacyclin, thromboxane, prolactin releasing hormone, lipotropin, brain natriuretic peptide, neuropeptide Y, histamine, endothelin, pancreatic polypeptide, renin, and enkephalin,

Growth factor refers to a naturally occurring protein capable of stimulating cellular proliferation and cellular differentiation. Examples are transforming growth factor beta (TGF-β), granulocyte-colony stimulating factor (G-CSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF-8), growth differentiation factor-9 (GDF9), acidic fibroblast growth factor (aFGF or FGF-1), basic fibroblast growth factor (bFGF or FGF-2), epidermal growth factor (EGF), and hepatocyte growth factor (HGF).

Antitumors or antineoplastics are drugs that inhibit and combat the development of tumors. Examples are actinomycin (e.g., actinomycin-D), anthracyclines (e.g., doxorubicin, daunorubicin, epirubicin), bleomycin, plicamycin, and mitomycin.

An anti-fungal agent is medication used to treat fungal infections. Examples are natamycin, rimocidin, filipin, nystatin, amphotericin B, miconazole, ketoconazole, clotrimazole, econazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, terconazole, terbinafine, amorolfine, naftifine, butenafine, anidulafungin, caspofungin, micafungin, benzoic acid, ciclopirox, flucytosine, griseofulvin, gentian violet, haloprogin, tolnaftate, undecylenic acid, tea tree oil, citronella oil, lemon grass, orange oil, palmarosa oil, patchouli, lemon myrtle, neem seed oil, coconut oil, zinc, and selenium.

Antiviral agents are a class of medication used specifically for treating viral infections. Examples are abacavir, aciclovir, acyclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, entry inhibitors (fusion inhibitor), famciclovir, fomivirsen, fosamprenavir, foscarnet, fosfonet, ganciclovir, gardasil, ibacitabine, immunovir, idoxuridine, imiquimod, indinavir, inosine, integrase inhibitor, interferon type III, interferon type TI, interferon type I, lamivudine, lopinavir, loviride, MK-0518 (raltegravir), maraviroc, moroxydine, nelfinavir, nevirapine, nexavir, nucleoside analogues, oseltamivir, penciclovir, peramivir, pleconaril, podophyllotoxin, protease inhibitor (pharmacology), reverse transcriptase inhibitor, ribavirin, rimantadine, ritonavir, saquinavir, stavudine, synergistic enhancer (antiretroviral), tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine.

Pain medications or analgesics (colloquially known as a painkiller) are members of the diverse group of drugs used to relieve pain. Examples are paracetamol/acetaminophen, nonsteroidal anti-inflammatory drugs (NSAIDs), COX-2 inhibitors (e.g., rofecoxib and celecoxib), morphine, codeine, oxycodone, hydrocodone, diamorphine, pethidine, tramadol, buprenorphine, tricyclic antidepressants (e.g., amitriptyline), carbamazepine, gabapentin and pregabalin.

An antihistamine is a histamine antagonist that serves to reduce or eliminate effects mediated by histamine, an endogenous chemical mediator released during allergic reactions. Examples are H1 antihistamine, aceprometazine, alimemazine, astemizole, azatadine, azelastine, benadryl, brompheniramine, chlorcyclizine, chloropyramine, chlorphenamine, phenylpropanolamine, cinnarizine, clemastine, cyclizine, cyproheptadine, dexbrompheniramine, dexchlorpheniramine, diphenhydramine, doxylamine, ebastine, emedastine, epinastine, fexofenadine, histamine antagonist (e.g., cimetidine, ranitidine, and famotidine; ABT-239, thioperamide, clobenpropit, impromidine, thioperamide, cromoglicate, nedocromil), hydroxyzine, ketotifen, levocabastine, mebhydrolin, mepyramine, mthapyrilene, methdilazine, olopatadine, pheniramine, phenyltoloxamine, resporal, semprex-D, sominex, talastine, terfenadine, and triprolidine.

Anti-inflammatory agent refers to a substance that reduces inflammation. Examples are corticosteroids, ibuprofen, diclofenac and naproxen, helenalin, salicylic acid, capsaicin, and omega-3 fatty acids.

Anti-infective agent is any agent capable of preventing or counteracting infection. It could be divided into several groups. Anthelminthics is one group of anti-infective agents comprising of albendazole, levamisole, mebendazole, niclosamide, praziquantel, and pyrantel. Another group is antifilarials, such as diethylcarbamazine, ivermectin, suramin sodium, antischistosomals and antitrematode medicine, oxamniquine, praziquantel, and triclabendazole. Another group is the antibacterials, which can be further subdivided. The beta lactam medicines are amoxicillin, ampicillin, benzathine benzylpenicillin, benzylpenicillin, cefazolin, cefixime, ceftazidime, ceftriaxone, cloxacillin, co-amoxiclav, imipenem/cilastatin, phenoxymethylpenicillin, and procaine benzylpenicillin. Other antibacterials are azithromycin, chloramphenicol, ciprofloxacin, clindamycin, co-trimoxazole, doxycycline, erythromycin, gentamicin, metronidazole, nitrofurantoin, spectinomycin, sulfadiazine, trimethoprim, and vancomycin. Examples of antileprosy medicines are clofazimine, dapsone, and rifampicin. Examples of antituberculosis medicines are amikacin, p-aminosalicylic acid, capreomycin, cycloserine, ethambutol, ethionamide, isoniazid, kanamycin, ofloxacin, pyrazinamide, rifampicin, and streptomycin. Examples of antifungal medicines are amphotericin B, clotrimazole, fluconazole, flucytosine, griseofulvin, nnystatin, potassium iodide. Antiviral agents are also anti-infective agents. An example of an antiherpes medicine is acyclovir. Examples of antiretrovirals are nucleoside/nucleotide reverse transcriptase inhibitors. Other examples are abacavir, didanosine, emtricitabine, lamivudine, stavudine, tenofovir disoproxil fumarate, zidovudine, non-nucleoside reverse transcriptase inhibitors, efavirenz, nevirapine, protease inhibitors, indinavir, lopinavir+ritonavir, nelfinavir, ritonavir, saquinavir and ribavirin. Examples of antiprotozoal medicines are antiamoebic and antigiardiasis medicines such as diloxanide, metronidazole; antileishmaniasis medicines such as amphotericin B, meglumine antimoniate, pentamidine; antimalarial medicines, such as amodiaquine, artemether, artemether+lumefantrine, artesunate, chloroquine, doxycycline, mefloquine, primaquine, quinine, sulfadoxine+pyrimethamine, chloroquine, and proguanil. Antipneumocytosis and antioxoplasmosis medicines are pentamindine, pyrimethamine, sulfamethoxazole+trimethoprim. Antitrypanosomal medicines are eflornithine, melarsoprol, pentamidine, suramin sodium, benznidazole, and nifurtimox. Antimigraine medicines, acetylsalicylic acid, paracetamol, and propranolol

Wound healing agents facilitate the body's natural process of regenerating dermal and epidermal tissue. Examples are fibrin, fibronectin, collagen, serotonin, bradykinin, prostaglandins, prostacyclins, thromboxane, histamine, neuropeptides, kinins, collagenases, plasminogen activator, zinc-dependent metalloproteinases, lactic acid, glycosaminoglycans, proteoglycans, glycoproteins, glycosaminoglycans (GAGs), elastin, growth factors (PDGF, TGF-β), nitric oxide, and matrix metalloproteinases, Examples of wound sealants are platelet gel and fibrin.

Cellular attractants or chemotaxic agents are chemicals or molecules in the environment that are sensed by bodily cells, bacteria, and other single-cell or multicellular organisms affecting their movements. Examples are amino acids, formyl peptides [e.g., N-formylmethionyl-leucyl-phenylalanine (fMLF or fMLP in references], complement 3a (C3a) and complement 5a (C5a), chemokines (e.g., IL-8); leukotrienes [e.g., leukotriene B4 (LTB4)].

Cytokines are groups of proteins and peptides that are signaling compounds produced by animal cells to communicate with one another. Cytokines can be divided into several families. Examples are the four alpha-helix bundle family with three subfamilies: the IL-2 subfamily [e.g., erythropoietin (EPO) and thrombopoietin (THPO)], the interferon (IFN) subfamily, the IL-10 subfamily. Other examples are the IL-1 family (e.g., IL-1 and IL-18), the IL-17 family, chemokines, immunoglobulin (Ig) superfamily, haemopoietic growth factor (type 1) family, Interferon (type 2) family, tumor necrosis factors (TNF) (type 3) family, seven transmembrane helix family, and transforming growth factor beta superfamily.

The surface or partial surface of the prefabricated biomedical device can be further treated by a physiochemical mean, a chemical mean, a coating mean, or a combination thereof to improve cellular attachment.

The surface of the prefabricated biomedical device can be treated with surface modification techniques pertaining to physiochemical means known in the art, such as, but not limited to, plasma or glow discharge, to improve the surface property of the construct for better cellular attachment.

The surface of the cell culture construct can be surface treated by chemical means, particularly with acids or bases. In a specific embodiment, the prefabricated biomedical device is treated with H₂SO₄, HNO₃, HCl, H₃PO₄, H₂CrO₄, or a combination thereof. In a specific embodiment, the prefabricated biomedical device is treated with NaOH, KOH, Ba(OH)₂, CsOH, Sr(OH)₂ Ca(OH)₂, LiOH, RbOH, or a combination thereof.

The surface of the cell culture construct can be further surface treated by coating means, which is applying a substance on the surface that is different from the material of the prefabricated biomedical device. The substance can be covalently bonded or physically absorbed to the surface of the struts and/or fibers. Alternatively, the substance can be bonded to the surface of the construct through hydrogen bonding, ionic bonding, Van der Waals force or a combination thereof. To increase the stability of the biological molecular coating, the coating can be cross-linked using various cross-linking technologies, such as chemical cross-linking, radiation, thermal treatment, or a combination thereof, etc. Further, the cross-linking can take place in a vacuum at an elevated temperature above room temperature. The radiation used for cross-linking can be e-beam radiation, gamma radiation, ultraviolet radiation, or a combination thereof.

The surface of the cell culture construct can be further surface coated with a synthetic polymer, such as, but not limited to, polyvinyl alcohol, polyethylene glycol, polyvinyl polypyrrolidone, poly(L-lactide), polylysine, etc.

The three dimensional porous cell culture construct can be coated with organic substance, such as gelatin, chitosan, polyacrylic acid, polyethylene glycol, polyvinyl alcohol, polyvinylpyrrilidone and a combination thereof.

In a specific embodiment, the prefabricated biomedical device is coated with an inorganic material, such as calcium phosphate, TiO₂, Al₂O₃, or a combination there of etc.

In a specific embodiment, the prefabricated biomedical device is coated with a composite coating of two or more organic materials, such as, but not limited to, gelatin and chitosan, polyacrylic acid and polyethylene glycol, polyvinyl alcohol and polyvinylpyrilidone, etc.

In a specific embodiment, the prefabricated biomedical device is coated with a composite of inorganic materials, such as calcium phosphate and TiO₂, calcium phosphate and Al₂O₃, etc. The inorganic composite coating is either chemically bonded to the surface, or physically absorbed to the surface of the said cell culture constructs.

In a specific embodiment, the prefabricated biomedical device is coated with a composite coating of inorganic and organic materials, such as, but not limited to, calcium phosphate/collagen, calcium phosphate/gelatin, calcium phosphate/polyethylene glycol, etc. The composite coating is either chemically bonded to the surface, or physically absorbed to the surface of the said cell culture constructs

### 5.3.1 Method of Making Hybrid Biomedical Device

The hybrid biomedical device can be fabricated using several methods, such as, but not limited to, directly culturing cells on the prefabricated biomedical device, or culturing cells seperately on the component parts of the medical device, and then assembling the ECM coated or non-coated parts together to obtain the final hybrid biomedical device. ECM coating process can be applied either in a cell culture vessel (cell culture plate, flask, etc) or in a biore actor.

### 5.3.1.1. Applying ECM Coating in a Static Cell Culture Condition

The present invention also provides methods of making a hybrid biomedical device by culturing living cells in a static cell culture condition within a tissue culture vessel, such as a polystyrene tissue culture plate. The prefabricated biomedical device can be a disc or cubic shape that fits into the well of a tissue culture plate. Cells can be seeded into the device using a dynamic seeding or static seeding method.

In one example using a static seeding method, a certain volume of cell suspension was pippetted onto the upper surface of the prefabricated device and allowed to attach for a certain time before flooding the scaffold and cells with medium. After being seeded with cells, this prefabricated device with seeded cells was maintained in the well plate submerged in growth medium, and cultured at 37° C. in an incubator in a 90% humidified atmosphere of 5-10% carbon dioxide in air.

In another example using a dynamic seeding method, cell seeding was performed by immersing the prefabricated device in cell suspension within a spinner flask, and then maintained at 37° C. in a humidified 5% CO₂ incubator. After seeding, cell culture constructs were placed into the wells of a tissue culture plate with medium for further culture at 37° C. in a humidified 5% CO₂ incubator. Culture medium was replaced regularly.

After cell culture was finished at certain time point, the cell cultured constructs were taken out of the cell culture plate and subjected to a dehydration process, such as a freeze drying process or an ethanol gradient dehydration process.

In the case where cells need to be removed, cells were trypsinized using a Trypsin-EDTA solution. The obtained hybrid biomedical device was rinsed with fresh serum-containing medium to inactivate the trypsin, followed by extensive rinses with neutral PBS.

### 5.3.1.2. Applying ECM Coating in a Dynamic Cell Culture Condition

The present invention also provides methods for using the cell culture construct to culture living cells within a bioreactor. The cell culture construct can be any size and shape that fits into the bioreactor.

An example of using a static seeding method is such that a certain volume of cell suspension was pipetted onto the surface of the prefabricated construct and allowed to attach for a certain period of time before flooding with medium. After being seeded with cells by either the static seeding or dynamic seeding method, these cell seeded devices were maintained in a bioreactor submerged in growth medium under dynamic conditions, and cultured at 37° C. in a 90% humidified atmosphere of 5-10% carbon dioxide in air. Culture medium was replaced regularly and constantly circulated through the bioreactor.

After cell culture was finished at certain time point, the hybrid biomedical devices were taken out of the bioreactor and subjected to a dehydration process.

In the case where the cells need to be removed, the cells were trypsinized using Trypsin-EDTA solution. The obtained hybrid biomedical device was rinsed with fresh serum-containing medium to inactivate the trypsin, followed by extensive rinses with neutral PBS.

### 5.4 Example 1

### Making a Hybrid Biomedical Device that has a Human Bone Matrix Like ECM Coating

Below is an example of coating a 3 dimensional biodegradable polycaprolactone (PCL) scaffold with a bone matrix like mineralized ECM coating

3D PCL (Sigma-Aldrich (St. Louis, Mo.)) scaffold was fabricated using 3D precision micro-fabrication technology, a layer by layer fabrication method. Within the scaffold, the struts within each layer were oriented 90° relative to the struts of the layer immediately below (FIG. 1.). Fiber diameter and spacing are approximately 300 μm and 500 μm, respectively. Scaffolds were sterilized by soaking in 70% ethanol for 1 hour and air dried in a bio-safety cell culture hood

Human bone marrow derived mesenchymal stem cells (hMSCs) (Lonza Walkersville, Inc (Cat#: PT-2501)) were re-suspended in MSCGM Basal Medium (Lonza Walkersville, Md.) with 10% fetal bovine serum (FBS), 2 mM of L-glutamine and 100 IU/ml penstrep. A density of cells at 0.1 million in 10 μl were seeded onto scaffold measuring 5.1 mm in diameter and 2.1 mm in height. The seeded scaffolds were then incubated for 3 hours to allow cell attachment. After that the scaffold was flooded with 200 μl of maintenance medium and kept in culture.

24 hours after cell seeding, culture media were replaced with osteogenic induction media, which contains basal medium supplemented with 50 μM ascorbic acid and 10 mM β-glycerophosphate. 0.1 μM of Dexamethasone was specifically used for hMSC osteogenic induction. Throughout the 4 weeks of in vitro culture, the induction media were changed every 2-3 days.

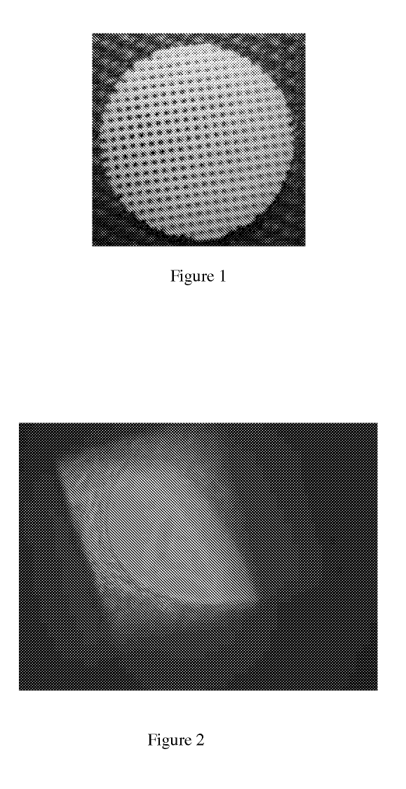

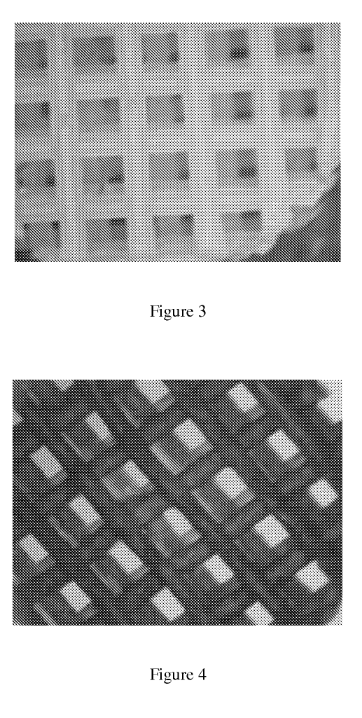

A layer of ECM secreted by cells was observed within the PCL scaffold (FIG. 2) under inverted light microscope. At day 7 and 28 of culture, the scaffolds were removed from culture medium and dehydrated using ethanol gradient. Von Kossa staining of hMSC culture derived ECM coating on the scaffolds indicated that cells grown on 3D PCL scaffold underwent more extensive mineralization at late stage of osteogenesis at day 28 (FIG. 4) than early stage at day 7 (FIG. 3).

### 5.5 Example 2

### Making a Hybrid Biomedical Device that has a ECM Coating Derived From Fibroblast Culturing

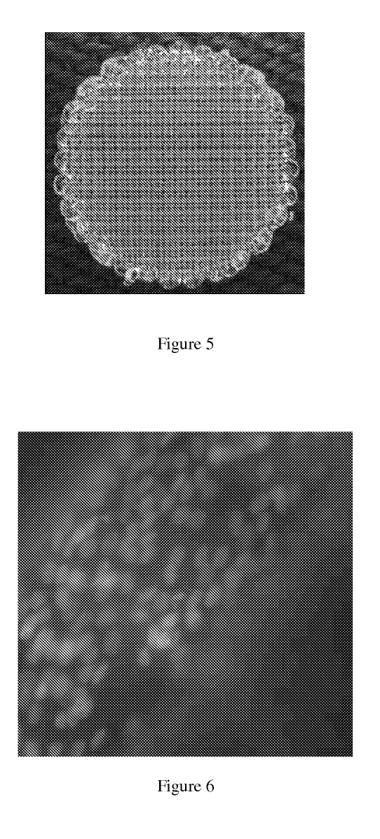

Polystyrene 3D scaffolds were used in this study (FIG. 5). Scaffolds were fabricated again using a precision 3D micro-fabrication method. NIH-3T3 fibroblasts, at a cell density of 0.1 million in 10 μl, were seeded onto 3D scaffolds measuring 5.1 mm in diameter and 2.1 mm in height. The seeded scaffolds were then incubated for 3 hours to allow for cell attachment. After that the scaffolds were flooded with 200 μl of fibroblast culture medium and kept in culture.

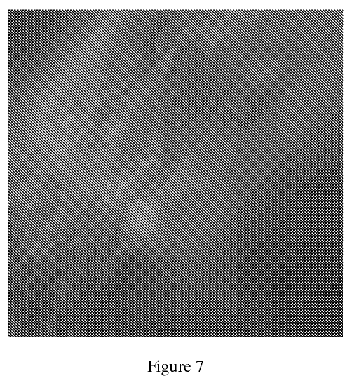

An ECM coating was formed in this culture environment. To further identify the composition of this cell cultured derived 3D tissue-like ECM structure formed by NIH-3T3 cells within the 3D PS scaffolds, the hybrid 3D scaffolds were fixed and stained with DAPI for viable nuclei (FIG. 6) and Alexa Fluor 488 phalloidin dye for F-actin microfilaments (FIG. 7). Fluorescent microscopy observation revealed that fibroblasts formed a 3D cell/ECM tissue like structure which covers PS scaffold fibers.

### 5.6 Example 3

### Making a Hybride Biomedical Device that Has a Rat Bone Matrix Like ECM Coating in a Bioreactor

The present invention also provides methods of using a porous cell culture construct for culturing living animal cells within a bioreactor. The cell culture construct used here was a disc shape (10 mm diameter discs with a thickness of 0.8 mm, porosity 80% and fiber diameter of 200 μm) that fit into the bioreactor.

Rat bone marrow stromal cells (MSCs) were statically seeded first onto the cell culture construct. 500 μl of MSC suspension with 250,000 rat MSCs was pipetted onto the upper surface of the porous cell culture construct, and allowed to attach for 2 hours at 37° C. before flooding with medium. After seeding with cells, these seeded cell culture constructs were maintained in a flow perfusion culture bioreactor. These cell seeded cell culture constructs were submerged in a complete osteo-differentiation medium, and cultured at 37° C. in a 90% humidified atmosphere of 5-10% carbon dioxide in air. The operation of the bioreactor system was driven by a peristaltic pump set at a rate of 1 ml/min. During the culture period, the culture medium was agitated to pass through the cell culture construct via the pores of the construct. Therefore, the cells were cultured under a dynamic shearing condition. The cells were cultured in the bioreactor for 4, 8, and 16 days, with a complete media exchange every 48 hours.

At the end of the culture period, the obtained hybrid constructs were rinsed with PBS and stored in 1.5 ml of distilled, deionized water at −20° C. until further use.

1. A hybrid biomedical device composed of a prefabricated biomedical device coated with an extracellular matrix (ECM) coating which is produced by an in vitro cell culture method.

2. The ECM coating is produced by culturing living cells on the prefabricated biomedical device in a cell culture vessel (flask, plate, Petri dish, bioreactor).

3. The prefabricated biomedical device in claim 1 has either a porous structure or a non-porous structure.

4. The prefabricated biomedical device in claim 1 is fabricated from a single type of synthetic material such as a type of polymer, a type of ceramic, and a type of metal.

5. The prefabricated biomedical device in claim 1 is fabricated from composites of materials, such as polymers, ceramics, metals and naturally derived biomaterials.

6. The cells used in claim 1 to produce ECM coating are genetically engineered which will selectively produce certain higher level of ECM components, such as higher level of collagen type I and fibronectin.

7. The cells used in claim 1 to produce ECM coating are transfected with certain DNA fragment to specifically produce higher level of growth factors or cytokines, such as BMP-2 and VEGF.

8. The cells used in claim 1 to produce ECM coating have mixed types of cell populations, such as whole bone marrow culture, or co-culture of fibroblasts and human umbilical vein endothelia cells, in order to produce more sophisticated tissue like structure or composition.

9. The prefabricated biomedical device in claim 1 has a pre-existing coating to facilitate the cellular attachment for in vitro cell culture.

10. The pre-existing coating in claim 8 is a naturally derived polymer coating, such as a collagen coating, a fibronectin coating, a glycosaminoglycan coating, a chitosan coating, a hyaluronic acid coating, etc

11. The pre-existing coating in claim 8 is a porous coating to facilitate the cellular attachment, proliferation and/or differentiation in the following cell culture coating process.

12. The pre-existing coating in claim 8 is also produced by an in vitro cell culture method.

13. The prefabricated biomedical device in claim 1 is impregnated with one or more biomolecules, such as protein, glycosaminoglycan, a naturally occurring compound, a therapeutic agent or a combination thereof.

14. The ECM coating in claim 1 is decellularized after the coating process is finished.

15. The ECM coating in claim 1 is in a dehydrated form.

16. The ECM coating in claim 1 has a porous structure.

17. The hybrid biomedical device in claim 1 is packaged and terminally sterilized.
